(19) United States
(12) Patent Application Publication
(10) Pub. No.: US 2025/0034511 A1
(43) Pub. Date: Jan. 30, 2025

(54) MYCELIUM-BASED LIGNOCELLULOSE COMPOSITE MATERIAL

(71) Applicant: RHEINISCH-WESTFÄLISCHE TECHNISCHE HOCHSCHULE (RWTH) AACHEN, Aachen (DE)

(72) Inventors: Dana Saez, Aachen (DE); Denis Grizmann, Aachen (DE); Anett Werner, Dresden (DE); Martin Trautz, Würselen (DE)

(21) Appl. No.: 18/709,209

(22) PCT Filed: Dec. 20, 2022
(86) PCT No.: PCT/EP2022/086923

§ 371 Date: May 10, 2024

(30) Foreign Application Priority Data
Dec. 21, 2021 (DE) 10 2021 134 036.5

(51) Int. Cl. C12N 1/14; C12R 1/645; E04C 1/40

(52) U.S. Cl. CPC C12N 1/14; E04C 1/40; C12R 2001/645

(57) ABSTRACT

The present invention is in the field of materials engineering and provides a method for producing a mycelium-based lignocellulosic composite material. Likewise, uses of the composite material according to the invention and the composite material itself are provided.

The present invention is in the field of material technique and provides a method for producing a mycelium-based lignocellulosic composite material. Likewise, uses of the composite material according to the invention and the composite material itself are provided.

The construction industry is responsible for more than a quarter of CO₂ emissions worldwide. In addition, 80-90% of these resources are used in the load-bearing structures of buildings. The demand for innovative materials that have beneficial properties and are used in load-bearing structures in particular is therefore high. In recent years, it has been shown that various materials can be produced based on mushroom mycelium. These materials have many advantages, such as good thermal insulation, low dry density and sound absorption, which make them suitable for use as building materials (e.g. as insulating material).

The production of these composite materials is based on the use of lignocellulosic substrates in combination with the natural growth of the vegetative component of the mycelium of filamentous fungi. When filamentous fungi grow, they form hyphae, which result in a close-meshed network and give the resulting material a solid structure.

Fungi belonging to the genus Basidiomycetes, such as Ganoderma lucidum, Ganoderma applanatum, Trametes hirsuta, Trametes versicolor or Fomes fomentarius, are mostly found in forests and fulfill the task of decomposing dead wood, among other things.

Wood consists of approx. 25-30% by weight lignin, 25-30% by weight pentosans (hemicellulose), 40-50% by weight cellulose as well as other components such as resins, terpenes, fats and fatty acids, proteins and minerals. Fungi can decompose lignin, hemicellulose and cellulose into their subunits. This occurs through the release of enzymes such as cellulases, laccases, amylases, proteases or lipases into the immediate environment, which break down the substrate. The degradation products are then absorbed by the hyphae and used for the growth of the fungus.

The state of the art describes several processes for the production of materials based on lignocellulose and fungal mycelium, as well as processes for influencing hyphal growth.

U.S. Pat. No. 9,914,906 B2 describes a process for cultivating mycelium on a lignocellulose substrate, which is enriched with additional nutrients. The resulting material is divided into individual particles, which are then used in various materials, such as bio-resins.

The European application EP 3 709 791 A1 deals with influencing the direction of growth of the mycelium. Here, the mycelium is cultivated in a special chamber into which a directed air flow is introduced. By varying the humidity and the air flow during cultivation, the direction of growth of the mycelium can be influenced so that the mycelium is homogeneously distributed in the resulting material. This process is used to produce materials that are intended to replace leather, textiles or foams.

U.S. Pat. No. 10,125,347 B2 also provides a method with which the expression of certain hyphal structures in fungi can be stimulated. This is done by bringing them into contact with other microorganisms that stimulate the filamentous fungi to form certain hyphal morphologies. The resulting material has a mycelium with reduced density.

European patent EP 2 702 137 B1 discloses a method for producing dried mycelium elements which can be combined as required. For this purpose, a mycelium element is produced in a first step as it is generally known. This element is then dried. If one now wants to connect the elements, at least one side of each of at least two mycelium elements is moistened and mixed with nutrient solution. The mycelium elements are then brought together on their moistened sides and the two elements are joined by the growth of the fungus. After a further drying step, in which the fungus is inactivated, the resulting elements can then be further processed. The authors describe this process as particularly suitable when a large area of material is required so that the smaller subunits can be transported and only assembled at their destination. No particular strength of the mycelium obtained is described.

The primary task of the present invention was therefore to provide a mycelium-based material which is particularly stable and has increased strength compared to known mycelium-based materials.

Further objects of the present invention are apparent from the following description and the appended patent claims.

The primary object of the present invention has been solved by providing a method for producing a mycelium-based lignocellulosic composite material comprising or consisting of the following steps:
a) Providing at least one lignocellulose-based substrate;
b) Inoculation of the substrate with fungal spores and/or fungal mycelium;
c) Mixing the inoculated substrate, preferably so that homogeneous growth of the mycelium is achieved,
d) incubating the obtained mixture from step c) in a first incubation phase for a period of between 5 and 7 days, at a temperature in the range from 20 to 28° C. and at a humidity in the range from 80 to 95%, in order to achieve cross-linked growth of the mycelium around the substrate;
e) filling the obtained incubated mixture from step d) into shape-conveying containers, which determine the shape of the basic unit of the composite material, and incubating the mixture in a second incubation phase for a time between 3 and 10 days, at a temperature in the range of between 20 to 30° C. and at a humidity in the range of between 80 to 95%, in order to achieve cross-linked growth of the mycelium around the substrate;
f) Obtaining at least one basic unit of the composite material with at least one joint interface;
g) providing at least two basic units obtained according to step e);
h) joining the at least two base units at the junction interface and incubating for a time between 10 and 30 days, at a temperature in the range of between 15 to 30° C. and at a humidity in the range of between 80 to 95%, to promote the formation of search and skeletal hyphae between the base units and obtaining a precursor of a mycelium-based lignocellulosic composite material;
i) drying the precursor of the mycelium-based lignocellulosic composite material at a temperature in the range of 65 to 90° C. and obtaining a mycelium-based lignocellulosic composite material with a residual moisture content of 10 to 12% by weight based on the total weight of the composite material.

In step a), firstly, the substrate is provided, which forms the basic framework for the growth of the fungus. This substrate is based on lignocellulose. Lignocellulose is the structural material in the cell wall of all woody plants and consists mainly of C5 and C6 sugars and lignin.

It is further preferred if the substrate provided in step a) is mixed with a nutrient solution, preferably in an amount of 20 g/L to 50 g/L. For example, the use of potato glucose broth (KGB) is preferred.

In the context of the present invention, it is preferred that the lignocellulosic-based substrate is in the form of coarse chips, particles or flour. In a particularly preferred embodiment, the substrate comprises or consists of—in each case based on the total weight of the substrate—40-60% by weight of chips having a size of 2-4 mm and 40-60% by weight of flour having a size of less than 2 mm.

Furthermore, it is preferred that the substrate has a moisture content of more than 10%, preferably more than 12% and particularly preferably 14%.

Preferably, the substrate is sterile. “Sterile” in the context of the present invention preferably means that the substrate is freed from any organisms that could negatively influence fungal growth by at least two treatments in an autoclave for a period of at least 60 minutes at a temperature of about 120° C.

If the substrate is not already provided sterile, but this is preferred in individual cases, a method according to the invention may comprise the following further step: Treatment in an autoclave for a period of at least 60 minutes at a temperature of about 120° C. or treatment with gamma irradiation.

In step b) of the method according to the invention, the preferably sterile substrate prepared in step a) is then inoculated with fungal spores and/or already growing fungal mycelium.

“Inoculation” in the context of the present invention means that the substrate is brought into contact with a defined quantity of fungal spores and/or fungal mycelium. Contact is preferably made by means of an inoculation loop. Alternatively, already growing fungal mycelium can be added to the substrate in the form of an already pre-incubated substrate (pre-culture). Furthermore, a mixture of fungal spores and already growing fungal mycelium can also be used.

Preferably, the substrate is inoculated with a quantity of fungal spores and/or fungal mycelium in a ratio of 1:100 (v/w) to 1:5 (v/w). The ratio depends on the form of inoculation material used (fungal spores and/or fungal mycelium).

This inoculated substrate is then mixed in step c) in such a way that the fungal spores and/or the fungal mycelium are homogeneously distributed over the entire substrate so that the subsequent growth is homogeneously distributed over the entire substrate.

The contained mixture is then subjected to a first incubation in step d).

“Incubating” in the context of the present invention means exposing the mixture to such conditions in which the fungal mycelium can grow or continue to grow. These are or comprise those described in step d).

Preferably, this first incubation step is carried out in bags, e.g. plastic bags (polypropylene filter bags), with a capacity of 3 to 8 liters.

Following the first incubation step, the incubated mixture is filled into shape-conveying containers that determine the shape of the basic unit of the composite material (step e)). The filled molds are incubated in a second incubation phase so that the fungal mycelium continues to grow.

At the end of the second incubation phase, at least one basic unit of the composite material with at least one joint interface is obtained (step f)).

A “joint interface” in the context of the present invention refers to the surface to which another base unit of the composite material can be attached.

In the subsequent step g), at least two basic units of the composite material are provided and then joined together in step h) so that the connecting interfaces of the base units are connected.

In a preferred embodiment, joining is achieved by placing the at least two basic units next to each other.

In a further embodiment, the joining can take place by exerting pressure on the two base units in the direction of the joint interfaces.

In one embodiment of the method, at least 3, preferably at least 4 and particularly preferably at least 10 basic units of the composite material are provided and joined together at their joint interfaces, e.g. such that the basic units are joined in series (with a corresponding number of joint interfaces).

The joined basic units are then incubated again in a third incubation phase so that the fungal mycelium grows in such a way that the two basic units of the composite material are joined.

Surprisingly, it was found that a manufacturing method according to the invention leads to the fungal mycelium forming predominantly skeletal hyphae at the joint interface of the composite material, which lead to increased strength of the material due to their morphology. The formation of such skeletal hyphae is shown schematically in FIG. 4. This increased strength of the material opens up new application possibilities that go beyond the applications described in the prior art as an insulating material or leather substitute.

In the last step i) of the method according to the invention, the composite material obtained in step h) is dried—preferably to constant mass—and a mycelium-based lignocellulose composite material with a residual moisture content of 10 to 15%, maximum 15%, preferably 10 to 12%, based on the total weight of the composite material is obtained. The residual moisture (and preferably, if required, the moisture content of the substrate (see above)) is preferably determined according to the principle of dielectric constants or by high-frequency measurement.

In a preferred embodiment, the present invention relates to a method wherein the junction interface of the at least two base units has a non-planar surface and wherein the junction interface is increased by a factor between 1.2 and 5, preferably between 1.3 and 2.5, compared to a planar surface.

Such an enlarged surface of the connecting surfaces of the base units is accompanied by non-planarity. This means that the surface of the connecting interface has elevations, which can be arranged symmetrically or asymmetrically.

A further preferred embodiment of the method according to the invention relates to a method as described herein, wherein the connecting interface of the at least two base units has an undulating longitudinal section.

In the context of the present invention, a “wavy” longitudinal section means that the surface of the connecting interface of the base units has elevations which have pointed (jagged) or round ends (rounded). These protrusions can follow one another directly or have at least one short planar section between the individual protrusions.

A further preferred embodiment relates to a method according to the invention, wherein the substrate is selected from the group consisting of birch wood, beech wood, cork, grass, straw, flax fibers, hemp fibers, oak wood, alder wood, willow wood or mixtures thereof, and preferably has a moisture content of at most 10% by weight, based on the total weight of the substrate. The moisture content is preferably determined as described above.

A further embodiment relates to a method according to the invention, wherein the fungal spores of step b) originate from one or more fungi selected from the group consisting of the class of Basidiomycetes, Ganoderma lucidum, Ganoderma applanatum, Fomes fomentarius, Trametes hirsuta, Trametes versicolor, Funalia trogii, Flammulina velutipes, Pleurotur sp., Pycnoporus sp., Lentinus edodes.

In a particularly preferred embodiment, the fungal spores and/or the fungal mycelium originate from Ganoderma lucidum and the substrate is beech wood.

Another aspect of the present invention relates to a composite material obtained or obtainable according to the method of the invention as described herein, preferably as described herein as preferred.

Preferably, the resulting composite material comprises or consists of 50 to 80% by weight lignocellulose and 20 to 50% by weight mycelium.

Further preferably, the composite material obtainable by a method according to the invention exhibits anisotropic behavior.

Particularly preferably, the composite material obtainable by a method according to the invention has a compressive strength of at least 1.5 N/mm², preferably at least 3 N/mm² and particularly preferably 6 N/mm², and/or the shear strength is at least 0.21 N/mm², preferably at least 0.22 N/mm² and particularly preferably 0.25 N/mm².

In a very preferred embodiment, the composite material obtainable by a method according to the invention has a shear strength of at least 1 N/mm², preferably at least 1.5 N/mm² and particularly preferably at least 2.5 N/mm².

The strength of a material is preferably determined by experimental test methods in accordance with or based on test standards (e.g. DIN-EN 826:2013, DIN-EN 12089:2013 or DIN-EN 12090:2013).

Yet another aspect of the present invention relates to a composite material comprising or consisting of
a) 50 to 80% by weight lignocellulose;
b) 20 to 50% by weight of mycelium.

A preferred embodiment of the composite material according to the invention relates to a composite material, wherein the composite material exhibits anisotropic behavior.

“Anisotropic behavior” in the sense of this text describes the behavior of a material when its physical, mechanical and chemical properties are directionally dependent. The material behavior is anisotropic if its elongation behavior and its strength are different parallel or transverse to a certain direction.

In the context of the present invention, the anisotropic behavior refers to the main direction in longitudinal extension along the skeletal hyphae formed at the joint interface. As a result, the material obtained has an advantageous force absorption.

In a further preferred embodiment of the composite material according to the invention, the compressive strength of the composite material is at least 1.5 N/mm², preferably at least 3 N/mm² and particularly preferably 6 N/mm², and/or the shear strength is at least 0.21 N/mm², preferably at least 0.22 N/mm² and particularly preferably 0.25 N/mm², preferably determined as described above.

In a very preferred embodiment of the composite material according to the invention, the shear strength is at least 1 N/mm², preferably at least 1.5 N/mm² and particularly preferably at least 2.5 N/mm².

A further aspect of the present invention relates to the use of a composite material obtainable by a method according to the invention or as described herein as a load-bearing or non-load-bearing building material, preferably as a load-bearing building material.

The increased strength and anisotropic behavior of the composite material allow it to be used in applications in which the material can absorb and transmit force.

In the following, the invention is described by illustrative, non-limiting examples.

### BRIEF DESCRIPTION OF THE FIGURES

FIG. 1 shows different shapes of the joint interfaces. The size of the joint interface is shown in Table 1.

FIG. 2 shows the force-displacement curve of the tested samples with different shapes of joint interfaces. The corresponding shear strengths are shown in Table 2.

FIG. 3 shows the growth process known from the prior art after 1 week (a), after two weeks (b) and after 3 weeks (c). Inoculated substrate consisting of coarse (2) and fine (3) wood chips is covered with an increasingly dense network of mycelial filaments (4). The dashed line symbolizes the material interface (1).

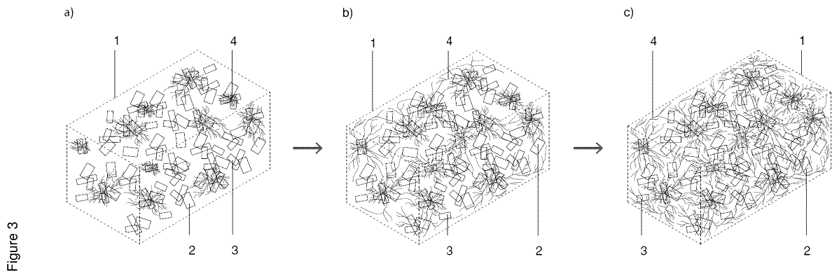

FIG. 4 shows the growth process according to the invention after one week (a), after two weeks (b), after three weeks (c) and after four weeks (d). Inoculated substrate consisting of coarse (2) and fine (3) wood chips is overgrown with mycelial filaments (4). When the base units are joined together at the connecting interface (5), a dense network of long mycelial filaments (skeletal hyphae) is formed. This condenses over time and leads to the increased strength of the material according to the invention. The dotted line symbolizes the material interface (1).

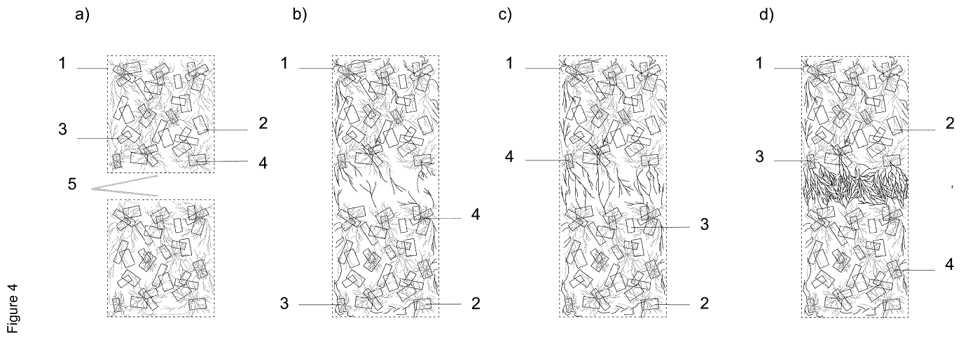

### EXAMPLES

### Pre-Culture

From a maximum four-week-old culture plate (KGB agar) with Ganoderma lucidum, 1 cm² large pieces of mycelium-grown Ganoderma lucidum were transferred sterilely into 150 ml KGB medium and then cultivated at 24° C. for seven days. Subsequently, the sterilized substrate was inoculated 1:10 (v/w) of the preculture.

### Inoculation/Cultivation in Bags

For cultivation in bags, autoclavable bags with integrated microfilters (SacO2) were used. At the beginning, the moisture content of 9% in the substrate was determined by drying at 105° C. for 24 h in order to be able to set the correct moisture level during cultivation of 65 to 70%.

Beechwood substrate was mixed with potato-glucose broth (KGB Fa. Roth) and stirred evenly. The amount of KGB to be used was determined by the residual moisture of the substrate. The substrate was completely homogenized, filled into the bags and then autoclaved. The bags were then inoculated 1:10 (v/w) with the preculture and incubated for five days at 24° C. under humid conditions in the incubator before the resulting mixture was filled into the forming containers. Pre-cultivation in the bag generally offers the advantage that the surface area is increased by breaking up the mycelium, thus stimulating apical growth of the hyphae, which leads to greater robustness.

### Cultivation in Shape-Conveying Containers

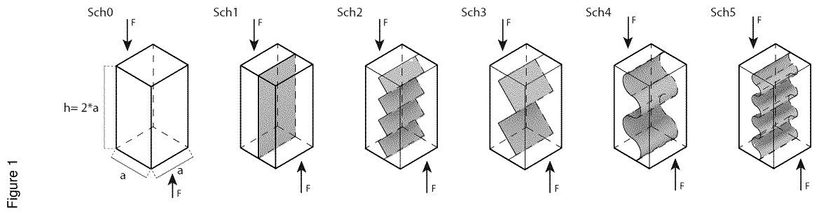

Five different shape-conveying containers were developed, each with a different shape of joint interface. The exact shapes and sizes of the joint interfaces can be seen in FIG. 1 and Table 1.

TABLE 1
Description of the surface and geometry of the joint interface of
the different samples (The volume of the sample is 134.4 cm3).

| Sample | Sh0 | Sh1 | Sh2 | Sh3 | Sh4 | Sch5 |
|---|---|---|---|---|---|---|
| Shape of the | — | planar | jagged | jagged | Rounded | rounded |
| connecting interface | | | small | big | large | small |
| Surface of the joint | 0 | 33.6 | 47.52 | 48.11 | 50.26 | 51.36 |
| interface [cm]² | | | | | | |

The resulting incubated mixture from the bags was separated or kneaded to avoid the formation of lumps and then filled into the shaping containers. All shaping containers were sterilized with ethanol before being filled with the mixture obtained from the bags. The shaping containers were then sealed with a layer of parafilm.

The temperature was set to 25° C. in an incubator and the humidity in the incubator was adjusted to 85-95% by adding containers of distilled water, which evaporated during the cultivation process. The pH of the beech substrate was tested after autoclaving and found to be 4.7. Cultivation was carried out for 7 days.

### Assembling the Basic Units

After reaching the desired growth volume in the shape-conveying containers, the obtained base units were removed from the shaping containers and then joined together with their joint interfaces. The joined base units were then incubated in a further incubation step in an incubator at 25° C. and a humidity of 85-95% for 14 days.

### Drying

After completing the assembly of the basic units, the precursors of the mycelium-based lignocellulose composite material were dried at a constant temperature to stop the growth process. Drying was carried out at 80° C. until constant weight was achieved. A constant weight was achieved after 8 to 10 hours of drying. A mycelium-based lignocellulose composite material was obtained as a product.

### Strength Measurement

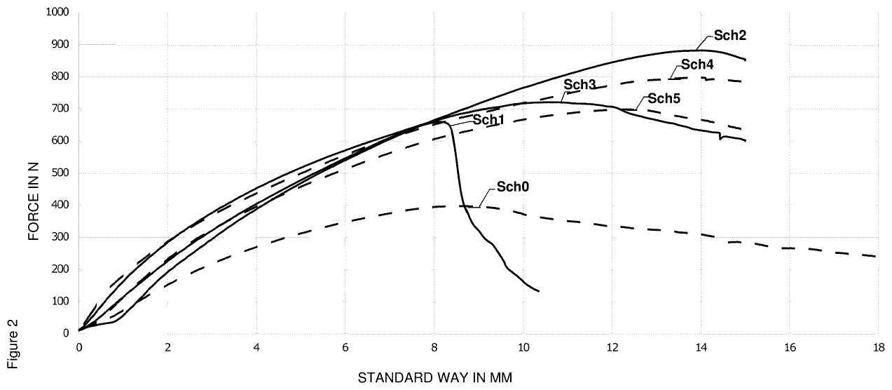

The materials obtained were then tested for their shear strength. Shear tests were carried out on samples Sch0 to Sch5 in accordance with DIN-EN 12090:2013. For each test, the force-displacement curve was recorded from which the shear strength was calculated (see FIG. 2). It is clearly visible that the different designs of the interfaces have an influence on the material behavior with regard to the type of failure as well as the stiffness and shear strength.

For example, specimen Sch1 with a planar joint interface shows an early onset of material fracture, which is represented in the force-displacement curve by a sharp drop in the curve. Furthermore, the comparison of the shear strengths shows that these are generally increased by the arrangement of an interface as well as by the enlargement of the joint interface.

The strengths of the material resulted in the following values (Table 2):

TABLE 2
Determined shear strengths of the tested samples.

| Sample | Sh0 | Sh1 | Sch2 | Sh3 | Sh4 | Sch5 |
|---|---|---|---|---|---|---|
| Strength | 0.124 | 0.206 | 0.275 | 0.225 | 0.249 | 0.218 |
| [N/mm²] | | | | | | |

1. A method for producing a mycelium-based lignocellulosic composite material comprising or consisting of the steps of:
a) Providing at least one lignocellulose-based substrate;
b) Inoculation of the substrate with fungal spores and/or fungal mycelium;
c) Mixing the inoculated substrate, preferably so that homogeneous growth of the mycelium is achieved,
d) Incubating the obtained mixture from step c) in a first incubation phase for a period of between 5 and 7 days, at a temperature in the range from 20 to 28° C. and at a humidity in the range from 80 to 95%, in order to achieve cross-linked growth of the mycelium around the substrate;
e) Filling the obtained incubated mixture from step d) into shaping containers, which determine the shape of the basic unit of the composite material, and incubating the mixture in a second incubation phase for a time between 3 and 10 days, at a temperature in the range of 20 to 30° C. and at a humidity in the range of 80 to 95%, in order to achieve cross-linked growth of the mycelium around the substrate;
f) Obtaining at least one basic unit of the composite material with at least one joint interface;
g) Providing at least two basic units obtained according to step e);
h) Joining the at least two base units at the junction interface and incubating for a time between 10 and 30 days, at a temperature in the range of 15 to 30° C. and at a humidity in the range of 80 to 95%, to promote the formation of search and skeletal hyphae between the base units and obtaining a precursor of a mycelium-based lignocellulosic composite material;
i) Drying the precursor of the mycelium-based lignocellulosic composite material at a temperature in the range from 65 to 90° C. and obtaining a mycelium-based lignocellulosic composite material with a residual moisture content of 10 to 15, preferably 10 to 12% by weight, based on the total weight of the composite material.

2. The method according to claim 1, wherein the connecting interface of the at least two basic units has a non-planar surface and wherein the connecting interface is enlarged by a factor of between 1.2 and 5, preferably between 1.3 and 2.5, compared to a planar surface.

3. The method according to claim 2, wherein the connecting interface of the at least two basic units has a wave-shaped longitudinal section.

4. The method according to claim 1, wherein the substrate is selected from the group consisting of birch wood, beech wood, cork, grass, straw, flax fibers, hemp fibers, oak wood, alder wood, willow wood and preferably has a moisture content of at most 10% by weight, based on the total weight of the substrate.

5. The method according to claim 1, wherein the fungal spores and/or the fungal mycelium from step b) originate from one or more fungi selected from the group consisting of the class of Basidiomycetes, Ganoderma lucidum, Ganoderma applanatum, Fomes fomentarius, Trametes hirsuta, Trametes versicolor, Funalia trogii, Flammulina velutipes, Pleurotur sp., Pycnoporus sp., Lentinus edodes.

6. The composite material obtained or obtainable by a method according to claim 1.

7. A composite material comprising
a) 50 to 80% by weight lignocellulose;
b) 20 to 50% by weight of mycelium.

8. The composite material according to claim 7, wherein the composite material exhibits anisotropic behavior.

9. The composite material according to claim 7, wherein the compressive strength of the composite material is at least 1.5 N/mm², preferably at least 3 N/mm² and particularly preferably 6 N/mm²
and/or
the shear strength is at least 0.21 N/mm², preferably at least 0.22 N/mm² and particularly preferably at least 0.25 N/mm².

10. Use of a composite material obtainable by the method according to claim 1 as a load-bearing or non-load-bearing building material, preferably as a load-bearing building material.

11. The composite material of claim 7, consisting of
a) 50 to 80% by weight lignocellulose;
b) 20 to 50% by weight of mycelium.
